(19) United States
(12) Patent Application Publication
(10) Pub. No.: US 2010/0120652 A1
(43) Pub. Date: May 13, 2010

(54) TREATMENT OF BED MITE AND BED BUG INFESTATIONS

(76) Inventor: Michael L. Corrado, Perkasie, PA (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/538,807

(22) Filed: Aug. 10, 2009

Related U.S. Application Data
(60) Provisional application No. 61/087,399, filed on Aug. 8, 2008.

(51) Int. Cl. C11D 3/48; A61K 31/7048; A61K 38/48; A61L 9/01

(52) U.S. Cl. 510/319; 514/30; 424/94.65; 424/76.8

(57) ABSTRACT

Methods for preventing or eradicating an infestation of bed mites or bed bugs in an article by contacting the article with an effective amount of an avermectin-containing composition. Avermectin-containing compositions used in the disclosed method are also described.

### CROSS-REFERENCE TO RELATED APPLICATION

The present invention claims priority benefit under 35 U.S.C. §119(e) of U.S. Patent Application Ser. No. 61/087,399 filed Aug. 8, 2008, the disclosure of which is incorporated herein by reference.

### FIELD OF THE INVENTION

This invention relates to methods of treating bed mite infestation of carpets, bedding, pillows and bed linens with the intent of decreasing allergic reactions in sensitized individuals and methods of treating bed bug infestations of bedding. The invention also related to products combining anti-parasitic compounds for killing the bed mites and bed bugs with proteases for denaturing the antigens from bed mite fecal pellets that produce the allergic reactions.

### BACKGROUND OF THE INVENTION

Among the most common causes of perennial, non-seasonal allergies in humans is the sensitization of humans to bed mites and their fecal deposits. Bed mites are microscopic arachnids which live within the first few centimeters of bedding, pillows, carpets or bed linens including sheets and comforters. These mites subsist by feeding on the desquamated human skin cells. Their fecal pellets contain proteins, and humans continuously inhale both mites and their pellets year round. Allergic reactions to these mites include sneezing, burning eyes, ear, eye and nose itching, congestion and cough.

Mites can be killed by exposure to direct sunlight, but carting of mattresses to the back yard is either impractical or impossible for high-rise apartment dwellers. Removing the fecal material without killing the mite is of no value. The weight of mites and their pellets over time can accumulate to be a quarter of the weight of the pillow.

Bed bugs were once a common scourge. Bed bugs are insects (not arachnids as are bed mites). Bed bugs bite the sleeping individual and feed on their blood or other bodily secretions. Bed bugs are returning to the United States and Western Europe as people from non-industrial countries immigrate or travel to these industrialized societies.

Avermectin is a natural fermentation product derived from the soil bacterium Streptomyces avermitilis. Avermectin is a highly effective anti-parasitic and insecticidal macrocyclic lactone that can paralyze and kill both bed mites and bed bugs by inhibiting glutamate-gated chloride ion channels. Other semi-synthetic forms of avermectin and mutated forms of Streptomyces avermitilis containing avermectin, for example doramectin, ivermectin, selamectin, and eprinomectin have found medicinal uses as well. Ivermectin, the most widely used synthetic avermectin, is the dihydro form of avermectin and is a highly effective anti-parasitic and insecticide.

Mammals, including humans, are impervious to these effects of avermectins by virtue of the P glycoprotein contained in the placental and blood brain barriers. For this reason ivermectin has enjoyed safe use in children, adults and pregnant women when given orally for the treatment of a variety of parasites, and oral dosage forms of ivermectin have enjoyed considerable success in the treatment of internal parasites.

### SUMMARY OF THE INVENTION

The present invention incorporates the discovery that ivermectin can be used environ-mentally to treat and kill both bed mites and bed bugs. Therefore, according to one aspect of the present invention, a method is provided for preventing or eradicating an infestation of bed mites or bed bugs in an article by contacting the article with an effective amount of an avermectin-containing composition. According to a more specific embodiment, a composition is applied to the surface of the article containing from about 0.1 to about 4% by weight of avermectin.

According to one embodiment of this aspect of the invention, the avermectin is an ivermectin. According to another embodiment of this aspect of the present invention, the article is a mattress or carpet. According to yet another embodiment of this aspect of the invention, the article is a fabric article such as clothing or bed clothes. According to another embodiment of the invention, the avermectin is formulated in an inert bulk powder, such as sodium bicarbonate, or as a liquid in an inert solvent. In a more specific embodiment, the solution contains water and a solubilizing or emulsifying agent. In an even more specific embodiment the solubilizing or emulsifying agent is a surfactant.

According to another embodiment of this aspect of the invention, the avermectin is contacted with the article for at least 15 minutes up to about 24 hours. According to a more specific embodiment the article is a carpet and the avermectin is contacted with the carpet for at least 15 minutes, after which the carpet is vacuumed to remove the avermectin product and any mites or insects. According to an even more specific embodiment, the avermectin is contacted with the carpet for at least 30 minutes; however contact as little as ten minutes has proven effective and may be employed.

According to an alternative more specific embodiment, the article is a mattress and the avermectin is contacted with the mattress for at least 12 hours, after which the mattress is vacuumed to remove any bed mites or bed bugs. According to an even more specific example of this alternative embodiment, the mattress is covered with a plastic bag after the avermectin is applied. In another even more specific embodiment the mattress is re-treated with avermectin within three to six months.

According to another alternative more specific embodiment, the article is a fabric article, such as clothing or bed clothes and the article is laundered by a method in which an amount of an avermectin-containing composition is added to the laundry water alone, or in addition to detergent so that the laundry water contains from about 0.1 up to about 4.0% by weight of avermectin. The avermectin may be added to a soak, wash or rinse cycle, and is preferably added to the soak cycle and removed by the wash and rinse cycles. The duration of contact may be the length of time the wash load spends in the respective cycle. Preferably the duration of contact is at least 15 minutes and more preferably at least 30 minutes. However, contact as little as ten minutes has proven effective and may be employed. In the alternative, the wash load may be soaked in the avermectin solution up to about 24 hours prior to washing, after which the wash water maybe changed, or detergent may be added followed by initiation of the wash cycle.

According to another embodiment of this aspect of the invention, the avermectin is formulated with a proteolytic enzyme to denature protein antigens found in mite fecal pellets. In a more specific embodiment the proteolytic enzyme is selected from bromelain, papain, actinidin and ficin. In another more specific embodiment, a weight ratio of protease to avermectin between about 1:10 and about 1:1 is used.

The present invention also provides avermectin-containing products for use in the methods according to the present invention. According to one aspect of the present invention, a composition is provided containing at least one avermectin and an inert carrier. The avermectin may be formulated as an additive for products conventionally used to clean or treat the article to which the avermectin is to be applied.

Products directly applied to the article will contain between about 0.1 to about 4.0 wt. % of one or more avermectins. Thus, a powdered or liquid carpet cleaner or carpet deodorizer may be formulated containing one or more avermectins at a concentration between about 0.1 to about 4.0% by weight. The same product can be used to clean or deodorize mattresses. Such products contain one or more ingredients selected from water, surfactants, deodorizers such as sodium bicarbonate, optical brighteners, perfumes, and the like. Cleaning products will contain higher levels of surfactants, but liquid deodorizing products will still contain surfactants to solubilize the avermectin.

Similarly, a powdered or liquid laundry product containing one or more avermectins can be formulated. Because such a product is not directly contacted with the article but diluted in laundry water, a higher concentration of avermectin should be employed. According to one embodiment the product contains between about 5 and about 25% by weight of avermectin and the quantity added to the laundry water is effective to provide a solution containing between about 0.1 and about 4% by weight of avermectin.

The laundry product may be formulated solely as an avermectin additive, or as a laundry detergent to be added to the wash cycle or a fabric softener to be added to the rinse cycle. Avermectin-impregnated dryer sheets may also be prepared. The laundry product may be a bulk powder or liquid product using the same inert ingredients conventionally found in powdered and liquid laundry products. Liquid products may also contain a surfactant added to form a solution or emulsion of the avermectin in the product. Typical additives include surfactants, builders, chelating agents, fabric softening agents, optical brighteners, perfumes, dyes, extenders, and the like.

According to one embodiment of this aspect of the invention, the avermectin products are formulated with a proteolytic enzyme for denaturing protein antigens found in mite fecal pellets. In a more specific embodiment the proteolytic enzyme is selected from bromelain, papain, actinidin and ficin. In another more specific embodiment, a weight ratio of protease to ivermectin between about 1:10 and about 1:1 is used.

A more complete appreciation of the invention and many other intended advantages can be readily obtained by reference to the following detailed description of the preferred embodiment and claims, which disclose the principles of the invention and the best modes presently contemplated for carrying them out.

### DETAILED DESCRIPTION OF PREFERRED EMBODIMENTS

This invention proposes the use of avermectins such as ivermectin to treat and kill both bed mites and bed bugs. In addition, proteolytic enzymes, i.e., proteases, may be added to denature the protein antigens found in mite fecal pellets. The avermectin or avermectin plus proteolytic enzyme may be applied topically to carpets or bedding or to the wash for pillows and bed linen.

The present invention will be described with reference to ivermectin. However, it should be understood that other avermectins can be substituted for ivermectin. The avermectin family is well-known and compounds suitable for use in the present invention are readily identified without undue experimentation.

When added to the wash, the ivermectin is preferably added to the soak cycle. The material is then washed out in the wash cycle. An amount of ivermectin effective to provide a concentration of ivermectin of 0.1% to 4% may be used in the soak cycle.

The ivermectin is preferably formulated as a higher concentration composition with inert bulk carriers typically used with powdered or laundry products with the instructions for adding the ivermectin taking into account the concentration of ivermectin in the bulk product. The ivermectin can also be formulated as a concentrated solution, with the instructions for use again taking into account the concentration of the ivermectin in the bulk product. The composition is then diluted to the 0.1 to 4.0% concentration by addition of an appropriate quantity to laundry water based on the concentration of ivermectin in the laundry product.

The laundry product may be formulated solely as an avermectin additive, or as a laundry detergent to be added to the wash cycle or a fabric softener to be added to the rinse cycle. The laundry product may be a bulk powder or liquid product using the same inert ingredients conventionally found in powdered and liquid laundry products. Liquid products may also contain a surfactant added to form a solution or emulsion of the avermectin in the product. Typical additives include surfactants, builders, chelating agents, fabric softening agents, optical brighteners, perfumes, dyes, extenders, and the like, one or more of which may be present in compounds according to the present invention.

The ivermectin may be formulated by itself or with an amount of a proteolytic enzyme effective to denature protein antigens found in mite fecal pellets. Proteolytic enzymes are well known and readily identified by those of ordinary skill in the art. According to one embodiment, a weight ratio of protease to ivermectin between 1:10 and 1:1 is used. In addition to methods of use, the present invention also includes compositions according to the present invention combining avermectins such as ivermectin with protease compounds.

Proteolytic enzymes commonly used as meat tenderizers are preferred. According to one embodiment of the invention, bromelain is used. Bromelain is composed of a number of protease enzymes and harvested commercially from the stems of pineapple plants, where it is concentrated. Besides being a meat tenderizer, bromelain is an excellent anti-inflammatory agent, blocking metabolites that cause swelling. Residual amounts remaining on bedding will help alleviate allergic reactions to mite fecal pellet antigens. Other suitable proteases commonly used as meat tenderizers that can be used include papain, extracted from the papaya, actinidin, extracted from kiwi, and ficin, extracted from figs.

When applied topically to surfaces, ivermectin or ivermectin plus a proteolytic agent is sprinkled, for example, over a carpet and left in place for at least 15 minutes up to about 24 hours before being vacuumed. The ivermectin can be applied as a liquid that is sprayed and permitted to dry or as a bulk powder that is dusted onto the surface of the object to be treated.

When applied to bedding, the ivermectin or ivermectin plus proteolytic agent is sprinkled over the entire surface of the bed. In an optional embodiment, a plastic cover is then applied to the bed overnight. Movement over the plastic cover will work the product into the bedding. The next day the cover is removed, and the material vacuumed from the mattress. A second plastic cover is applied over-night and the following day the mattress thoroughly vacuumed again. This process is repeated every three months for a year, then every six months thereafter.

Topical products are formulated containing the 0.1 to 4.0% ivermectin concentration intended for direct contact with a surface to be treated. The product can be a simple ivermectin-containing powder of liquid, or the product can be formulated as a carpet cleaner or deodorizer that can also be applied to mattresses. Such products contain one or more ingredients selected from water, surfactants, deodorizers such as sodium bicarbonate, optical brighteners, perfumes, and the like. Cleaning products will contain higher levels of surfactants, but liquid deodorizing products, as well as the simple avermectin-containing products, will still contain surfactants to solubilize the avermectin.

The present invention thus provides a simple and effective means by which bed mites and bed bugs may be removed from a sleeping environment such as a residence, hotel, motel, dormitory, campground, hospital, nursing home, and the like. The foregoing description of the preferred embodiment should be taken as illustrating, rather than as limiting, the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

What is claimed is:

1. A method for preventing or eradicating an infestation of bed mites or bed bugs in an article comprising contacting the article with an effective amount of an avermectin-containing composition.

2. The method of claim 1, wherein said composition comprises from about 0.1 to about 4% by weight of avermectin.

3. The method of claim 1, wherein the avermectin is an ivermectin.

4. The method of claim 1, wherein the avermectin is contacted with the article for at least 15 minutes up to about 24 hours.

5. The method of claim 1, wherein the article is a mattress or carpet.

6. The method of claim 5, wherein the article is a carpet, and said avermectin is contacted with said carpet for at least 30 minutes, after which said carpet is vacuumed to remove said avermectin and any mites or insects.

7. The method of claim 5, wherein the article is a mattress and said avermectin is contacted with said mattress for at least 12 hours, after which the mattress is vacuumed to remove any bed mites or bed bugs.

8. The method of claim 7, wherein said mattress is covered with a plastic bag after the avermectin is applied.

9. The method of claim 8, wherein said mattress is re-treated with avermectin within three to six months.

10. The method of claim 1, wherein the article is a fabric article.

11. The method of claim 10, wherein said fabric article is contacted with said avermectin by contacting said fabric article with laundry water containing from about 0.1 up to about 4.0% by weight of avermectin.

12. The method of claim 11, wherein said avermectin is added to the laundry water of the soak cycle of a washing machine, and the fabric article is soaked in avermectin-containing water up to about 24 hours prior to washing.

13. The method of claim 1, wherein said avermectin-containing composition further comprises a proteolytic enzyme.

14. The method of claim 13, wherein said proteolytic enzyme is selected from the group consisting of bromelain, papain, actinidin and ficin.

15. The method of claim 13, wherein the weight ratio of proteolytic enzyme to avermectin is between about 1:10 and about 1:1.

16. An avermectin-containing composition comprising at least one avermectin, a proteolytic enzyme, and an inert carrier.

17. The composition of claim 16, comprising between about 0.1 to about 4.0 wt. % of one or more avermectins.

18. The composition of claim 16, comprising between about 5 and about 25% by weight of one or more avermectins.

19. The composition of claim 16, wherein said one or more avermectins comprise at least one ivermectin.

20. The composition of claim 16, wherein said proteolytic enzyme is selected from the group consisting of bromelain, papain, actinidin and ficin.

21. The composition of claim 20, wherein the weight ratio of proteolytic enzyme to avermectin is between about 1:10 and about 1:1.

22. A powdered or liquid cleaning or deodorizing composition comprising one or more avermectins at a concentration between about 0.1 to about 4.0% by weight and one or more ingredients selected from the group consisting of water, surfactants, deodorizers, bulk fillers and extenders, optical brighteners and perfumes.

23. The composition of claim 22, wherein said one or more avermectins comprise at least one ivermectin.

24. The composition of claim 22, further comprising at least one proteolytic enzyme

25. The composition of claim 24, wherein said proteolytic enzyme is selected from the group consisting of bromelain, papain, actinidin and ficin.

26. The composition of claim 24, wherein the weight ratio of proteolytic enzyme to avermectin is between about 1:10 and about 1:1.

27. A powdered or liquid laundry composition comprising between about 5 and about 25% by weight of avermectin and one or more ingredients selected from the group consisting of surfactants, builders, chelating agents, fabric softening agents, optical brighteners, perfumes, dyes, bulk fillers and extenders.

28. The composition of claim 22, wherein said one or more avermectins comprise at least one ivermectin.

29. The composition of claim 27, further comprising at least one proteolytic enzyme

30. The composition of claim 29, wherein said proteolytic enzyme is selected from the group consisting of bromelain, papain, actinidin and ficin.

31. The composition of claim 29, wherein the weight ratio of proteolytic enzyme to avermectin is between about 1:10 and about 1:1.
